SEQUENCE LISTING (20 sequences)

SEQ ID NO: 1 - DNA, 5 bp, Artificial Sequence
  Consensus pentapyrimidine element
ycctt

SEQ ID NO: 2 - DNA, 10 bp, Artificial Sequence
  Sequence required to clone PCR products using vaccinia topoisomerase
nnnnaagggc

SEQ ID NO: 3 - DNA, 10 bp, Artificial Sequence
  Sequence generated from amplification procedure
gcccttnnnn

SEQ ID NO: 4 - DNA, 34 bp, Artificial Sequence
  loxP sequence
ataacttcgtatagcatacattatacgaagttat

SEQ ID NO: 5 - DNA, 34 bp, Artificial Sequence
  loxP511 sequence
ataacttcgtatagtatacattatacgaagttat

SEQ ID NO: 6 - DNA, 34 bp, Artificial Sequence
  loxC2 sequence
acaacttcgtataatgtatgctatacgaagttat

SEQ ID NO: 7 - DNA, 34 bp, Artificial Sequence
  Flp Recombination Target Site
gaagttcctattctctagaaagtataggaacttc

SEQ ID NO: 8 - DNA, 14 bp, Artificial Sequence
  TP901-1 sequence
ttcaatyaaggtaa

SEQ ID NO: 9 - DNA, 10 bp, Artificial Sequence
  TnpX sequence
gcccngargg

SEQ ID NO: 10 - DNA, 12 bp, Artificial Sequence
  R4 sequence
gaagcagtggta

SEQ ID NO: 11 - DNA, 3 bp, Artificial Sequence
  phiC31 sequence
ttg

SEQ ID NO: 12 - DNA, 6 bp, Artificial Sequence
  lambda integrase sequence
caagtt

SEQ ID NO: 13 - DNA, 6 bp, Artificial Sequence
  HK022 integrase sequence
aacctt

SEQ ID NO: 14 - DNA, 5 bp, Artificial Sequence
  PCR forward primer
aaggg

SEQ ID NO: 15 - DNA, 7 bp, Artificial Sequence
  5′ overhang sequence
cgaaggg

SEQ ID NO: 16 - DNA, 12 bp, Artificial Sequence
  Topoisomerase recognition sites
cccttgcaaggg

SEQ ID NO: 17 - DNA, 470 bp, Artificial Sequence
  Donor vector with topoisomerase I recognition sites
gaggattgggcccttgatcaagggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaacccccttctagaagggctatctcggtct

SEQ ID NO: 18 - DNA, 58 bp, Artificial Sequence
  Acceptor vector
aaaaagctcccccttgatcaagggcgggagcttgcccttctagaagggtatatccatt

SEQ ID NO: 19 - DNA, 468 bp, Artificial Sequence
  Recombinant vector
aaaaagctcccccttgatcaagggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaacccccttctagaagggtatatccatt

SEQ ID NO: 20 - DNA, 11 bp, Artificial Sequence
  3′ end of PCR product used for topoisomerase-based transfer
gcccttnnnnn

(19) United States
(12) Patent Application Publication
(10) Pub. No.: US 2005/0181417 A1
(43) Pub. Date: Aug. 18, 2005

(54) System for the rapid manipulation of nucleic acid sequenaces

(75) Inventors: David J. Miles, San Diego, CA (US); Lyle C. Turner, Cardiff, CA (US); Robert Marcil, Carlsbad, CA (US); Gina C. McConnell, San Diego, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation, Carlsbad, CA (US)

(21) Appl. No.: 11/080,810

(22) Filed: Mar. 16, 2005

Related U.S. Application Data
(62) Division of application No. 10/368,442, filed on Feb. 20, 2003.
Application No. 10/368,442 is a continuation of application No. 09/804,653, filed on Mar. 12, 2001, now abandoned.
Application No. 09/804,653 is a continuation of application No. 09/141,935, filed on Aug. 28, 1998, now abandoned.

(51) Int. Cl. C12Q 1/68; C12P 19/34

(52) U.S. Cl. 435/6; 435/91.1

(57) ABSTRACT

The present invention is a cell-free subcloning system utilizing three elements: (1) a donor vector that contains a nucleic acid sequence to be transferred to another vector flanked by a site-specific recombination sequence and one or more optional additional nucleic acid sequences, (2) an acceptor vector that contains a site-specific recombination sequence and one or more optional additional nucleic acid sequences, and (3) a site-specific recombinase that recognizes the site-specific recombination sequences in the donor and acceptor vectors so as to transfer the transfer sequence from the donor to the acceptor vector upon contact of the three elements of the system. Also disclosed are rapid subcloning methods employing the vectors and enzymes disclosed herein and kits for use in such methods.

### FIELD OF THE INVENTION

The invention disclosed herein relates to the field of molecular biology and methods useful therefor. More particularly the invention relates to methods for subcloning of nucleic acid sequences.

### BACKGROUND OF THE INVENTION

The discovery and isolation of restriction endonucleases, specific enzymes capable of manipulating nucleic acid sequences, precipitated a revolution in molecular biological techniques. Restriction endonucleases were used to cut large DNAs into smaller fragments that could be re-attached to heterologous pieces of DNA by ligases. These techniques allowed scientists to transfer a gene encoding a particular protein into a relatively small plasmid vector that could be transfected into a cell for production of the encoded protein.

Over the years, a large number of vectors have been developed for a wide variety of specialized research, manufacturing, and production uses. For example, many types of expression vectors have been developed that allow heterologous proteins to be expressed in an increasingly larger number of cell types, including insect, plant, mammalian, and bacterial cells. Among expression vectors, specialized vectors have been developed that facilitate large scale production of proteins, for instance, by increasing levels of the protein produced or by introducing elements into the protein that aid in purification. Other vectors have been designed for use in specific research protocols, such as conducting one-hybrid or two-hybrid screens. Each specialized vector contains a specific set of nucleic acid sequences that give it its particular features. No one vector can contain all of these features, however, as the vector would eventually become too large to be easily manipulated. Thus, a nucleic acid sequence of interest must be moved from one vector to another as different specialized needs arise, a process known as subcloning.

Conventional subcloning methods require that each vector into which a nucleic acid sequence is to be subcloned contain restriction endonuclease recognition/digestion sites that are absent in the nucleic acid sequence in order to prevent the nucleic acid sequence from being cut into one or more pieces when subjected to the restriction endonuclease for removal from the vector and passage to the next vector. One must, therefore, either know the entire sequence of the nucleic acid being subcloned or test it with each restriction endonuclease proposed for use to see if it contains a matching recognition site. Either process requires time and resources to perform.

In addition, conventional subcloning methods require that the nucleic acid sequence being subcloned have sequences at its 5′ and 3′ ends that match the restriction endonuclease site into which it is being inserted. As not all available vectors have the same restriction endonuclease sites, the nucleic acid sequence to be transferred must usually be modified at its ends to make it compatible with each vector to be used in subcloning techniques.

Another drawback to conventional subcloning techniques is the use of ligases. These enzymes are relatively slow acting, require ATP, and generally are highly temperature sensitive.

A need still exists in the art, therefore, for a simple, rapid system for the manipulation of nucleic acid sequences between vectors. The present invention addresses that need.

### BRIEF DESCRIPTION OF THE INVENTION

The present invention comprises a cell-free subcloning system, methods for the rapid manipulation and subcloning of nucleic acid sequences using the system, and kits suitable for use in conducting such methods. In general, the system, methods, and kits of the invention utilize three elements. The first element is a donor vector comprising (1) a transfer sequence of nucleic acid to be transferred to an acceptor vector, (2) a site specific recombination nucleic acid sequence flanking the transfer sequence as shown in FIG. 1A; and, (3) optionally, one or more additional nucleic acid sequences. The second element is an acceptor vector comprising (1) a site-specific recombination sequence that matches the site-specific recombination sequence of the donor vector as shown in FIG. 1B, and (2) one or more additional nucleic acid sequences. The third element is a site-specific, ATP independent recombinase, that recognizes the site specific recombination sequences in both the donor and acceptor vectors.

The site-specific recombinases employed in the practice of the present invention are enzymes that spontaneously recognize and cleave at least one strand of a double strand of nucleic acids within a sequence segment known as the site-specific recombination sequence. In the donor vector, the site specific recombination sequences are placed contiguously on either side of (i.e., “flank”) a transfer sequence of nucleic acid whose excision from the donor vector and transfer to the acceptor vector is desired. In use, the donor vector containing the transfer sequence and the acceptor vector are placed within a single cell-free solution. Upon addition of the site-specific recombinase to the cell-free solution, the transfer sequence is excised from the donor vector. In some portion of the acceptor vectors in the cell-free solution (i.e., “occasionally”) the excised transfer sequence is ligated into the acceptor vector by operation of the recombinase upon the site-specific recombination sequence, without the use of a separate ligase to accomplish the ligation. The acceptor vectors generally further comprise a selectable marker gene to aid in identifying and isolating from the cell-free solution using known methods those acceptor vectors into which the transfer sequence has been successfully inserted. The site-specific recombination sequences of the donor and acceptor vehicles are preferably identical, but can vary in nucleic acid sequence so long as recognition of the site-specific recombination sequence by the recombinase is preserved despite the variance.

The present invention thus affords a novel single-step method and associated vectors and kits for moving nucleic acid sequences, such as recombinant DNA molecules, from one type of subcloning vector to another that overcomes the above-described problems in the art. For example, the invention eliminates the need for incorporation of “add on” base sequences to transfer sequence to provide unique restriction sites.

In particular, topoisomerase-based cloning circumvents any problems associated with addition of nontemplated nucleotides by DNA polymerase at the 3′ end of the amplified DNA. Any nontemplated base (N) at the 3′ end of a PCR product destined for topoisomerase-based transfer (GCCCTTxxxxN-3′) will dissociate spontaneously upon covalent adduct formation, and will therefore have no impact on the ligation to vector. Second, the only molecule that can possibly be ligated into the acceptor vector is the covalently activated transfer sequence and the transfer sequence can only be transferred to the acceptor vector. There is no potential for in vitro covalent closure of the acceptor vector itself, which ensures low background. There is also no opportunity for the transfer sequences to ligate to one another, which precludes cloning of concatameric repeats. In addition, unintended internal restriction of an uncharacterized sequence is avoided because the use of common restriction enzymes is avoided.

### DESCRIPTION OF THE FIGURES

FIG. 1 illustrates the cell-free system of the invention.

FIG. 1A is a double stranded nucleic acid sequence representing a donor vector with a double stranded nucleic acid transfer sequence flanked by topoisomerase I recombinase recognition sites (single underlined) with a 4 base core sequence (within brackets).

FIG. 1B is a double stranded nucleic acid representing an invention acceptor vector containing two recombinase recognition sites that match those in the donor vector and 10 base pair spacer sequences (double underlined) ready to receive a transfer sequence.

FIG. 1C is a double stranded nucleic acid representing a new recombinant vector created by the operation of topoisomerase I upon the donor and acceptor vectors of FIGS. 1A and 1B, respectively. The transfer sequence is now inserted into the acceptor vector.

FIG. 2 is schematic representation of the method of the invention utilizing a donor vector (“pDonor”) containing a selectable marker gene other than Zeocin, an origin of replication sequence (“ori”), and a transfer sequence (“gene of interest”) flanked by lox P recognition sites. The acceptor vector (“pAcceptor”) contains a gene encoding resistance to the antibiotic Zeocin™ (“Zeo”), an origin of replication sequence (“ori”), and a gene encoding ccdB, a lethal compound, flanked by loxP sites. The arrow indicates that when the donor and acceptor vectors are combined in a reaction mixture in the presence of the recombinase Cre, a new recombinant vector (“pRecombinant”) is created, which recombinant vector contains the transfer sequence and a gene encoding Zeo. Cells transformed with the reaction mixture will grow in the presence of the antibiotic Zeocin™ only if the recombination event has successfully occurred.

### DETAILED DESCRIPTION OF THE INVENTION

In one embodiment of the invention, there is provided a cell-free subcloning system comprising (1) a donor vector comprising a transfer sequence flanked by site-specific recombination sequences, (2) an acceptor vector comprising a site-specific recombination sequence that matches the site-specific recombination sequences of the donor vector, and (3) a site-specific recombinase capable of recognizing the site-specific recombination sequence Each vector is of duplex nucleic acid sequence, and the transfer is a bivalent strand transfer. In the acceptor vector, the transfer sequence will be inserted in the immediate vicinity of and downstream of, or can be adjacent to, the site-specific recombination sequence Optionally, one or more additional nucleic acid sequences, such as a selection marker gene, an origin of replication, a promoter-enhancer sequence, and the like can be included in the donor and acceptor vectors. The subcloning event occurs in a cell-free environment without the need to use restriction enzyme(s), and the transfer of the transfer sequence to the acceptor vector occurs without the expense of ATP.

In a presently preferred embodiment of the invention, following the site-specific recombination event that occurs between the site-specific recombination sequences located on each vector (i.e., the donor and acceptor vectors), the transfer sequence is inserted into the acceptor vector in a manner that retains the proper translational reading frame of the transfer sequence.

As used herein “vector” means a recombinant nucleic acid sequence of duplex DNA that has been constructed to comprise one or more functional units not found together in nature. Examples include circular, double-stranded, extrachromosomal DNA molecules (plasmids), cosmids (plasmids containing COS sequences from lambda phage), viral genomes comprising non-native nucleic acid sequences, and the like. When used in the context of describing a vector, the terms “donor” and “acceptor” refer to the fact that one vector (the donor) will contain a nucleic acid sequence, referred to herein as the “transfer sequence,” that is to be excised and transferred to another (the acceptor) vector. Any given vector can be a donor or an acceptor, depending on whether it is the vector from which a nucleic acid sequence is being transferred, or the vector into which a nucleic acid sequence is introduced.

Both donor and acceptor vectors contain site-specific recombination sequences, which are sequences of nucleic acids that are specifically recognized by a particular site-specific recombinase. Site specific recombinases, as the term is used herein, are enzymes that catalyze the excision and/or recombination of nucleic acid sequences, and may form intermediate complexes with the transfer sequence DNA during the recombination event. These enzymes recognize a relatively short, unique recognition and recombination. Recombinases particularly useful in the practice of the invention are those that function in a wide variety of cell types because such enzymes do not require any host specific factors and do not require ATP to function. Examples of site-specific recombinases of this type include type I topoisomerases (S. Shuman, J. Biological Chemistry 266:11372-79, 1991), integrases (Argos, et al., EMBO J. 5:433-440, 1986), resolvases (Hallet and Sherratt, FEMS Microbiol. Rev. 21:157-178, 1997), and the like.

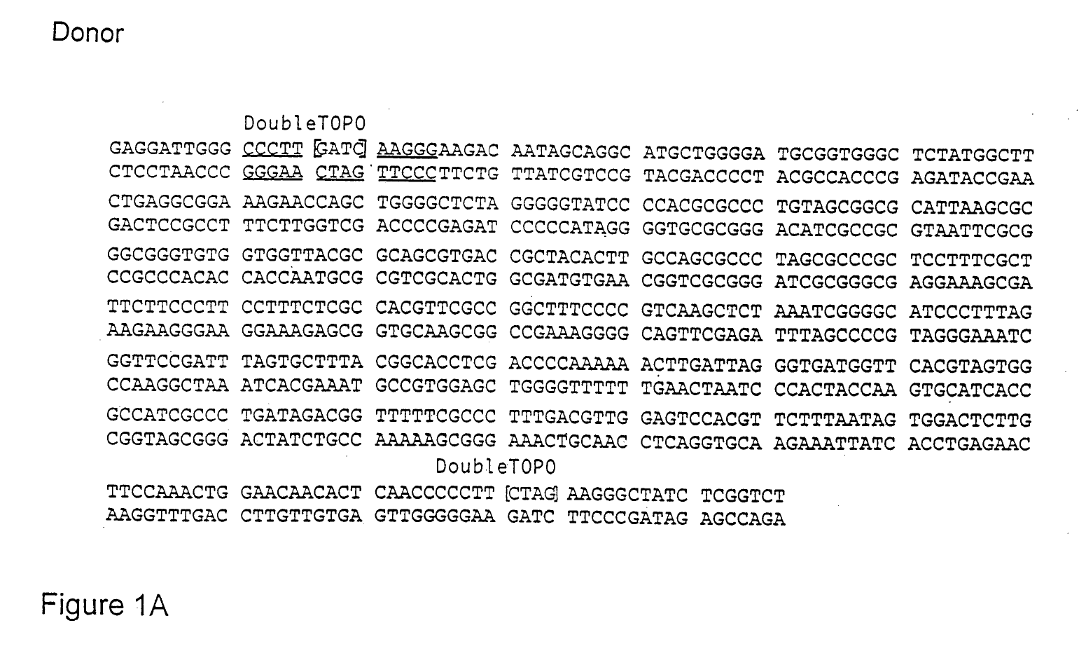

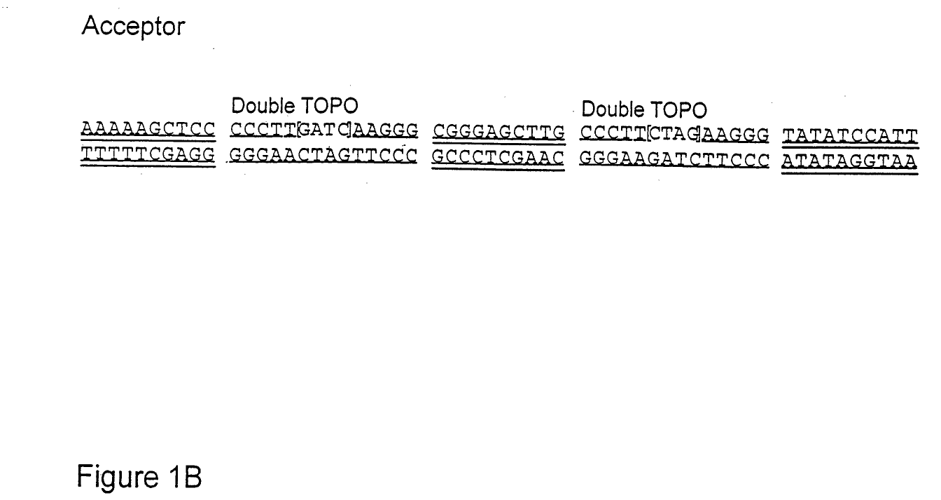

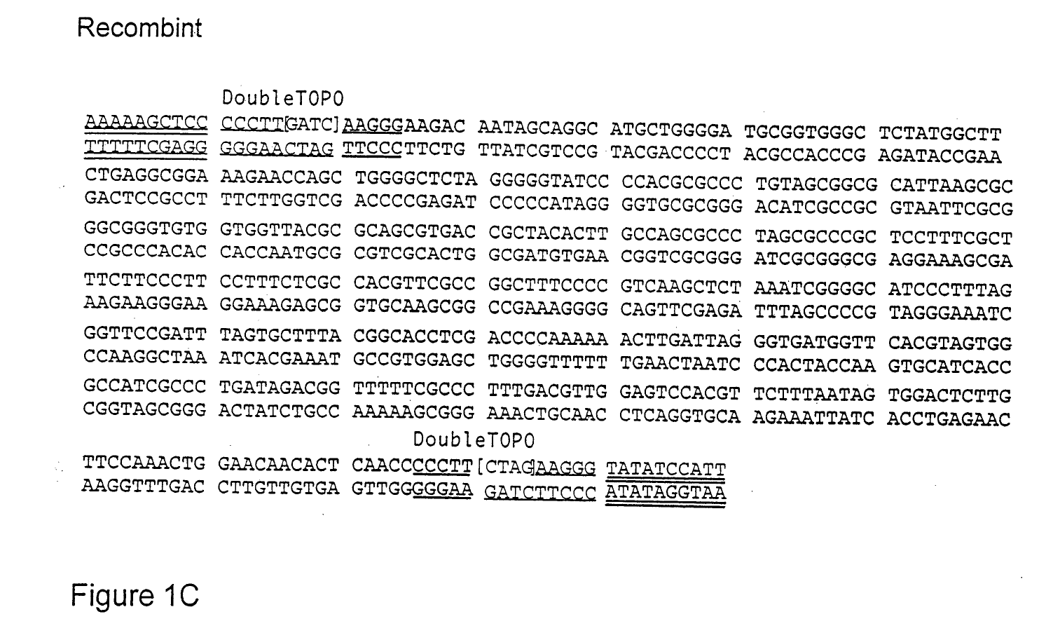

A particularly suitable enzyme for use in the practice of the invention is a type I topoisomerase, particularly vaccinia DNA topoisomerase. Vaccinia DNA topoisomerase binds to duplex DNA and cleaves the phosphodiester backbone of one strand. The enzyme exhibits a high level of sequence specificity, akin to that of a restriction endonuclease. Cleavage preferentially occurs at a consensus pentapyrimidine element 5′-(C/T)CCTT↓(SEQ ID NO: 1) in the scissile strand. In the cleavage reaction, bond energy is conserved via the formation of a covalent adduct between the 3′ phosphate of the incised strand and a tyrosyl residue of the topoisomerase I protein. Vaccinia topoisomerase can religate the covalently held strand across the same bond originally cleaved (as occurs during DNA relaxation) or it can ligate the strand to a heterologous acceptor DNA 5′ end containing a site specific recombination site, such as the DNA in the invention acceptor vector, and thereby create a new recombinant molecule, as shown in FIG. 1C.

When the substrate is configured such that the scissile bond with the topoisomerase is situated near (within about 10 to about 12 base pairs of) the 3′ end of a DNA duplex, cleavage is accompanied by the spontaneous dissociation of the downstream portion of the cleaved strand in the donor vector. The resulting topoisomerase-DNA complex, containing a 5′ single-stranded tail, can religate to an acceptor DNA if the acceptor molecule has a 5′ OH terminated acceptor strand with sequence (e.g. of at least a four base overhang) complementary to that of the activated donor complex (i.e., the single strand tail of the noncleaved donor strand in the immediate vicinity of the sissile phosphate). In the absence of an acceptor strand, the topoisomerase can transfer the CCCTT strand to water, releasing a 3′-phosphate-terminated hydrolysis product, or to glycerol. However, the hydrolysis reaction is much slower than religation to an acceptor DNA strand of the acceptor vector, the extent of strand transfer to non-DNA nucleophiles being generally about 14-40%.

The specificity of vaccinia topoisomerase in DNA cleavage and its versatility in strand transfer have inspired topoisomerase-based strategies for polynucleotide synthesis in which DNA oligonucleotides containing CCCTT cleavage sites serve as activated linkers for the joining of other DNA molecules with compatible termini (S. Shuman, J. Biol. Chem. 269:32678-32684, 1994). The use of vaccinia topoisomerase type I for cloning generally is described in detail in U.S. Pat. No. 5,766,891, which is incorporated by reference herein in its entirety.

Bivalent strand transfer also results in circularization of the acceptor vector DNA by placing the topoisomerase cleavage sites on the transfer sequence (a synthetic bivalent substrate) and cloning the cleaved DNA into the donor vector. This strategy is well-suited to the cloning of DNA fragments amplified by PCR. To clone PCR products using vaccinia topoisomerase, it is preferred to include a 10 nucleotide sequence-5′-XXXXAAGGGC-(SEQ ID NO:2) at the 5′ end of the two primers used for amplification. The 5′-XXXX segment can correspond to any 4-base overhang that is compatible with the restriction site into which the PCR product will ultimately be cloned. The amplification procedure will generate duplex molecules containing the sequence 5′-GCCCTTxxxx-3′(SEQ ID NO:3) at both 3′ ends (where xxxx is the complement of XXXX). Incubation of the PCR product with topoisomerase will result in cleavage at both termini and allow the covalently activated PCR fragment to be ligated into the donor vector DNA. From the donor vector the transfer sequence can be simultaneously transferred to one or a number of different acceptor vectors engineered to contain functional sequences suitable for accomplishing different types of cloning procedures. For example, an acceptor vector that is a bacterial expression vector generally includes a promoter (such as the lac promoter), the Shine-Dalgarno sequence (for transcription initiation) and the start codon (AUG). Similarly, a eukaryotic expression vector includes, but is not limited to, a heterologous or homologous promoter for RNA polymerase II, a downstream polyadenylation signal, the start codon AUG, and a termination codon for detachment of the ribosome.

The donor complex formed upon cleavage by topoisomerase at a 3′ proximal site is extremely stable. The transfer sequence can be transferred nearly quantitatively to an acceptor vector with a complementary site even after many hours of incubation of the covalent topo-DNA complex at room temperature. The topo-transfer sequence complex can even be denatured with 6 M guanidine HCl and then renatured spontaneously upon removal of guanidine with complete recovery of strand transferase activity. Thus, a topoisomerase-activated vector can be prepared once in quantity and used as many times as needed for preparation of various types of acceptor vectors according to the invention.

In addition, two major families of site-specific recombinases from bacteria and unicellular yeast have been described: the integrase family and the resolvase/invertase family. In these recombinases, strand exchange catalyzed by site specific recombinases occurs in two steps of (1) cleavage and (2) rejoining, involving a covalent protein-DNA intermediate formed between the recombinase enzyme and the DNA strand(s). The nature of the catalytic amino acid residue of the enzyme and the line of entry of the nucleophile is different for these two recombinase families. For cleavage catalyzed by the invertase/resolvase family, the nucleophile hydroxyl is derived from a serine and the leaving group is the 3′-OH of the deoxyribose. For the integrase family, the catalytic residue is a tyrosine and the leaving group is the 5′-OH. In both recombinase families, the rejoining step is the reverse of the cleavage step.

The recombinase activity of Cre has been studied as a model system for the integrases. Cre is a 38 kD protein isolated from bacteriophage P1. It catalyzes recombination at a 34 base pair stretch of nucleic acids called loxP. The loxP site has the sequence 5′-ATAACTTCGTATAGCATACATTATACGAAGTTAT-3′ (SEQ ID NO: 4; spacer region underlined), consisting of two 13 base pair palindromic repeats flanking an eight basepair core sequence (Hoess et al., Proc. Natl. Acad. Sci USA 79:3398, 1982 and U.S. Pat. No. 4,959,217, the disclosure of which is herein incorporated by reference in its entirety). The repeat sequences act as Cre binding sites with the crossover point occurring in the internal spacer core. Each repeat appears to bind one protein molecule wherein the DNA substrate (one strand) is cleaved and a protein-DNA intermediate is formed having a 3′-phosphotyrosine linkage between Cre and the cleaved DNA strand. Crystallography and other studies suggest that four proteins and two loxP sites (each on a different DNA molecule) form a synapsed structure in which the DNA resembles models of four-way Holliday-junction intermediates, followed by the exchange of a second set of strands to resolve the intermediate into recombinant products (see, Guo, et al., Nature 389:40-46, 1997). The asymmetry of the core region of the loxP recombination sequence is responsible for directionality of the recombination reaction. When two loxP sites on the same DNA molecule are in a directly repeated orientation, Cre excises the DNA between these two sites, leaving a single loxP site on the DNA molecule (Abremski et al., Cell 32:1301, 1983). Thus, the repeat sequences act as Cre-specific binding sites with the recombination crossover point occurring in the core.

The loxP site is so complex in size that it occurs only in the PI phage genome. Therefore, use of the loxP sites in the invention vectors assures that the enzyme will not cut the transfer sequence within the interior of the sequence unless the transfer sequence is from the PI phage genome. The activity of Cre in a wide variety of cellular backgrounds, including yeast, shows that Cre does not require host specific factors for activity (Sauer Mol. Cell. Biol. 7:2087-2096, 1987), plants (Albert et al., Plant J. 7:649-659, 1995; Dale and Ow, Gene 91:79-85, 1990; Odell et al., Mol. Gen. Genet. 223:369-378, 1990) and mammals, including both rodent and human cells (van Deursen et al., Proc. Natl. Acad. Sci. USA 92:7376-7380,1995; Agah et al., J. Clin. Invest. 100:169-179, 1997; Sauer and Henderson, New Biologist 2:441-449, 1990).

The Cre protein also recognizes a number of variant or mutant lox sites (variant relative to the loxP sequence), including the loxB, loxL and loxR sites, which are found in the E. coli chromosome. Other variant lox sites include loxP511 (5′-ATAACTTCGTATAGTATACATTATACGAAGTTAT-3′ (SEQ ID NO:5; spacer region underlined); loxC2 (5′-ACAACTTCGTATAATGTATGCTATACGAAGTTAT-3′ (SEQ ID NO:6; spacer region underlined; U.S. Pat. No. 4,959,217). Additional variants of the loxP site can be prepared by those of skill in the art and will generally have no more than a total of one to three point mutations in the two repeats that comprise the site-specific recombination sequence. Cre catalyzes the cleavage of the lox site within the spacer region and creates a six base-pair staggered cut. The two 13 bp inverted repeat domains of the lox site represent binding sites for the Cre protein. The two lox sites may differ so long as Cre is able to recognize both lox sites. However, if two lox sites differ in their spacer regions in such a manner that the overhanging ends of the cleaved DNA cannot reanneal with one another, Cre cannot efficiently catalyze a recombination event using the two different lox sites. The efficiency of the recombination event will depend on the degree and the location of the variations in the binding sites. For example, the loxC2 site can be efficiently recombined with the loxP site because the two lox sites differ by a single nucleotide in the leftbinding site. Thus, when Cre is the site specific recombinase used in the practice of the invention methods, the site-specific recombination sequence is a loxP site, or a variant thereof recognized by the Cre enzyme.

A recombinase of the integrase family with similar function is Flp, a recombinase identified in strains of Saccharomyces cerevisiae that contain 2β-circle DNA. Flp recognizes a DNA sequence consisting of two 13 basepair inverted repeats flanking an 8 basepair core sequence (5′-GAAGTTCCTATTCTAGAAAGTATAGGAACTTC-3′ (SEQ ID NO: 7); spacer underlined) called FRT (Flp Recombination Target site). A third repeat follows at the 3′ end in the natural sequence, but does not appear to be required for recombinase activity. The Flp gene has been cloned and expressed in E coli and in mammalian cells (PCT International Patent Application PCT/US92/01899, Publication No: WO 92/15694, the disclosure of which is herein incorporated by reference) and has been purified (Meyer-Lean et al., Nucleic Acids Res. 15:6469, 1987; Babineau et al., J. Biol. Chem. 260:12313, 1985; Gronostajski and Sadowski, J. Biol. Chem. 260:12328, 1985).

Like Cre, Flp is functional in a wide variety of systems including bacteria (Huang et al., J. Bacteriology 179:6076-6083, 1997), insects (Golic and Lindquist, Cell 59:499-509, 1989; Golic and Golic, Genetics 144:1693-1711, 1996), plants (Lyznik et al., Nucleic Acids Res 21:969-975, 1993) and mammals (U.S. Pat. Nos. 5,677,177 and 5,654,182), which shows the Flp does not require host specific factors for operability.

Unlike the integrases, each member of the resolvase subfamily of recombinase enzymes contains an N-terminal catalytic domain having a high degree (>35%) of sequence homology among the subfamily members (Crellin and Rood, J. Bacteriology 179(16):5148-5156, 1997; Christiansen et al., J. Bacteriology 178(17):5164-5173, 1996). Despite this, like the integrases, many of the resolvases do not require host specific accessory factors (Thorpe and Smith, PNAS USA 95:5505-5510, 1998).

Other site-specific recombinases suitable for use in the system and methods of the present invention include RecA (Ferrin et al., PNAS USA 95:2156-57,1998), HK022 integrase, lambda integrase (with or without Xis), which recognizes Att sites (Weisberg et al., In: Lambda II, Hendrix et al., Eds., Cold Spring Harbor Press, Cold Spring Harbor, N.Y., 1983), and the like.

The process of strand exchange used by the resolvases is somewhat different than the process used by the integrases. The resolvases usually make cuts close to the center of the crossover site, and the top and bottom strand cuts are often staggered by 2 basepairs, leaving recessed 5′ ends. A protein-DNA linkage is formed between phosphodiester from the 5′ DNA end and a conserved serine residue close to the amino terminus of the recombinase. Like the invertases, two proteins units are bound at each crossover site, however, no equivalent to the Holliday-junction intermediate is formed (see Stark et al., Trends in Genetics 8(12:432-439, 1992, incorporated by reference herein).

The nucleic acid sequences recognized as recombination sites by members of the resolvase family differ in several ways from the integrases. The sites used for recognition and recombination of the phage and bacterial DNAs (the native host system) are generally non-identical, although they typically have a common core region of nucleic acids. The bacterial sequence is generally called the AttB sequence (bacterial attachment) and the phage sequence is called the AttP sequence (phage attachment). Because AttB and AttP are somewhat different sequences, recombination will result in a stretch of nucleic acids (called AttL or AttR for left and right) that is neither an AttB sequence nor an AttP sequence, and is probably unrecognizable as a recombination site to the relevant enzyme, thus reducing the possibility that the enzyme will catalyze a second recombination reaction that would reverse the first.

The individual resolvases and the nucleic acid sequences that they recognize have been less well characterized than Cre and Flp, although most of the core sequences have been identified. The core sequences of some of the resolvases useful in the practice of the invention include TP901-1-5′-TTCAAT(T/C)AAGGTAA (SEQ ID NO: 8); TnpX-5′-GCCCNGA(G/A)GG (SEQ ID NO: 9), R4-5′-GAAGCAGTGGTA (SEQ ID NO: 10), and φC31-5′-TTG (SEQ ID NO: 11) (see Rausch and Lehmann, NAR 19:5187-5189, 1991; Shirai et al., J. Bacteriology 173(13):4237-4239, 1991; Crellin and Rood, J Bacteriology 179:5148-5156, 1997; Christiansen et al., J Bacteriology 176:1069-1076, 1994, all of which are incorporated by reference herein.)

In general, Site-specific recombination sequences of the invention vary in length, although they are generally less than 50 nucleotides. Particularly suitable site-specific recombination sequences include the recognition sequences for vaccinia topoisomerase I (5′-(C/T)CCTT↓, SEQ ID NO: 1), Cre (5′-ATAACTTCGTATA GCATACAT TATACGAAGTTAT-, SEQ ID NO: 4), Flp (5′-GAAGTTCCTATAC TTCTAGAA GAATAGGAACTTC, SEQ ID NO: 7), lambda integrase (5′-CAAGTT, SEQ ID NO: 12), HK022 integrase (5′-AACCTT, SEQ ID NO: 13), and the like. The present invention is illustrated, but not limited by the use of vectors containing topoisomerase I sites.

Any nucleic acid sequence is suitable as a transfer sequence as long as there is a desire for the sequence to be moved from one vector to another. The transfer sequence may, for example, encode a protein, peptide or functional RNA (such as antisense sequences, hammerhead ribozymes, and the like). A transfer sequence encoding a protein or peptide may be either a gene sequence or a coding sequence. As used herein, a “gene sequence” is the entire nucleic acid sequence that is necessary for the synthesis of a functional polypeptide or RNA molecule; whereas a “coding sequence” is limited to the nucleic acids encoding the amino acid sequence of a protein.

The transfer sequence may also be a sequence whose function, if any, is not yet known, such as an expressed sequence tag (EST) fragment. Such sequences can be used as diagnostic probes, or as aids in the identification and cloning of a larger sequence containing the EST fragment.

The vectors employed in the practice of the invention contain one or more nucleic acid sequences in addition to the site-specific recombination sequences, and transfer sequence in the case of a donor vector. The additional nucleic acid sequences will generally have some function in the replication or integrity of the vector, in the expression of a protein, in the modification of an expressed protein, and the like. Particularly useful nucleic acid sequences include promoter-enhancer sequences, selection marker sequences, origins of replication, inducible element sequences, fusion protein producing sequences, for example, localization signal sequences, epitope tags, proteolytic cleavage recognition sequences, polypeptides that facilitate purification, and the like.

Promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA polymerase. Eukaryotic promoters are more complex. Most promoters utilized in vectors are transcribed by RNA polymerase II. General transcription factors (GTFs) first bind specific sequences near the start and then recruit the binding of RNA polymerase II. In addition to these minimal promoter elements, small sequence elements are recognized specifically by modular DNA-binding/trans-activating proteins (e.g. AP-1, SP-1) that regulate the activity of a given promoter. Viral promoters serve the same function as bacterial or eukaryotic promoters and either provide a specific RNA polymerase in trans (bacteriophage T7) or recruit cellular factors and RNA polymerase (SV40, RSV, CMV). Viral promoters may be preferred as they are generally particularly strong promoters.

Promoters may be, furthermore, either constitutive or regulatable (i.e., inducible or derepressible). Inducible elements are DNA sequence elements which act in conjunction with promoters and bind either repressors (e.g. lacO/LAC Iq repressor system in E. coli) or inducers (e.g. gal1/GAL4 inducer system in yeast). In either case, transcription is virtually “shut off” until the promoter is derepressed or induced, at which point transcription is “turned-on”.

Examples of constitutive promoters include the int promoter of bacteriophage λ, the bla promoter of the β-lactamase gene sequence of pBR322, the CAT promoter of the chloramphenicol acetyl transferase gene sequence of pPR325, and the like. Examples of inducible prokaryotic promoters include the major right and left promoters of bacteriophage (P_{L} and P_{R}), the trp, reca, lacZ, LacI, AraC and gal promoters of E. coli, the a-amylase (Ulmanen et al, J. Bacteriol. 162:176-182, 1985) and the sigma-28-specific promoters of B. subtilis (Gilman et al., Gene Sequence 32:11-20, 1984), the promoters of the bacteriophages of Bacillus (Gryczan, In: The Molecular Biology of the Bacilli, Academic Press, Inc., NY, 1982), Streptomyces promoters (Ward et al., Mol. Gen. Genet. 203:468-478, 1986), Pichia promoters (U.S. Pat. Nos. 4,855,231 and 4,808,537), and the like. Exemplary prokaryotic promoters are reviewed by Glick (J. Ind. Microbiol. 1:277-282, 1987); Cenatiempo (Biochimie 68:505-516, 1986); and Gottesman (Ann. Rev. Genet. 18:415-442, 1984).

Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-5955, 1984), the CMV promoter, the EF-1 promoter, Ecdysone-responsive promoter(s), tetracycline-responsive promoter, and the like.

Selection marker sequences are valuable elements in expression vectors as they provide a means to select for growth only those cells which have been successfully transformed with a vector containing the selection marker sequence and express the marker. Such markers are of two types: drug resistance and auxotrophic. A drug resistance marker enables cells to detoxify an exogenously added drug that would otherwise kill the cell. Auxotrophic markers allow cells to synthesize an essential component (usually an amino acid) while grown in media which lacks that essential component.

Common selectable marker gene sequences include those for resistance to antibiotics such as ampicillin, tetracycline, kanamycin, bleomycin, streptomycin, hygromycin, neomycin, Zeocin™, and the like. Selectable auxotrophic gene sequences include, for example, hisD, which allows growth in histidine free media in the presence of histidinol.

A further element useful in a vector is an origin of replication sequence. Replication origins are unique DNA segments that contain multiple short repeated sequences that are recognized by multimeric origin-binding proteins and which play a key role in assembling DNA replication enzymes at the origin site. Suitable origins of replication for use in expression vectors employed herein include E. coli oric, colE1 plasmid origin, 2μ and ARS (both useful in yeast systems), sf1, SV40 EBV oriP (useful in mammalian systems), and the like.

Fusion protein producing sequences may be included in a vector employed in the present invention. When two protein-coding sequences not normally associated with each other in nature are in the same reading frame the resulting expressed protein is called a “fusion protein” as two distinct proteins and/or fragments have been “fused” together. Fusion proteins have a wide variety of uses. For example, two functional enzymes can be fused to produce a single protein with multiple enzymatic activities or short peptide sequences can be fused to a larger protein and serve as aids in purification or as means of identifying expressed protein by serving as epitopes detectable by specific antibodies. Thus examples of fusion protein producing sequences useful in the vectors of the invention include epitope-tag encoding sequences, affinity purification-tag encoding sequences, functional protein encoding sequences, and the like.

Epitope tags are short peptide sequences that are recognized by epitope specific antibodies. A fusion protein comprising a recombinant protein and an epitope tag can be simply and easily purified using an antibody bound to a chromatography resin. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

Affinity purification tags are generally peptide sequences that can interact with a binding partner immobilized on a solid support. Preferably, the recombination event in the invention method places the transfer sequence in frame with the sequence encoding the affinity domain, so that the affinity purification tag and the expression product of the transfer sequence is expressed as a fusion protein when the sequence is expressed. DNA sequences encoding multiple consecutive single amino acids, such as histidine, when fused to the expressed protein, may be used for one-step purification of the recombinant protein by high affinity binding to a resin column, such as nickel sepharose. An endopeptidase recognition sequence can be engineered between the polyamino acid tag and the protein of interest to allow subsequent removal of the leader peptide by digestion with enterokinase, and other proteases. Sequences encoding peptides, such as the chitin binding domain (which binds to chitin), glutathione-S-transferase (which binds to glutathione), biotin (which binds to avidin and strepavidin), and the like, can also be used for facilitating purification of the protein of interest. The affinity purification tag can be separated from the protein of interest by methods well known in the art, including the use of inteins (protein self-splicing elements, Chong et al., Gene 192:271-281, 1997).

The use of the term “functional protein encoding sequence”, as used herein, indicates that the fusion protein producing element of a vector encodes a protein or peptide having a particular activity, such as an enzymatic activity, a binding activity, and the like. For example, a functional protein encoding sequence may encode a kinase catalytic domain (Hanks and Hunter, FASEB J 9:576-595, 1995), producing a fusion protein that can enzymatically add phosphate moieties to particular amino acids, or may encode a Src Homology 2 (SH2) domain (Sadowski, et al., Mol. Cell. Bio. 6:4396, 1986; Mayer and Baltimore, Trends Cell. Biol. 3:8, 1993), producing a fusion protein that specifically binds to phosphorylated tyrosines.

The foregoing elements can be combined to produce vectors suitable for use in the methods of the invention. Those of skill in the art would be able to select and combine the elements suitable for use in any particular system.

Suitable prokaryotic vectors include plasmids such as those capable of replication in E. coli (for example, pBR322, ColE1, pSC101, PACYC 184, itVX, pRSET, pBAD (Invitrogen, Carlsbad, Calif.), and the like). Such plasmids are disclosed by Sambrook (cf Molecular Cloning: A Laboratory Manual, second edition, edited by Sambrook, Fritsch, & Maniatis, Cold Spring Harbor Laboratory, 1989). Bacillus plasmids include pC194, pC221, pT127, and the like, and are disclosed by Gryczan (In: The Molecular Biology of the Bacilli, supra, pp. 307-329). Suitable Streptomyces plasmids include p1J1O1 (Kendall et al., J. Bacteriol. 169:4177-4183,1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Suitable eukaryotic plasmids include, for example, BPV, EBV, vaccinia, SV40, 2-micron circle, pcDNA3.1, pcDNA3.1/GS, pYES2/GS, pMT, p IND, pIND(Sp1), pVgRXR (Invitrogen), and the like, or their derivatives. Such plasmids are well known in the art (Botstein et al., Miami Wntr. Symp. 19:265-274, 1982; Broach, In: The Molecular Biology of the Yeast Saccharomyces: Life Cycle and Inheritance, Cold Spring, Harbor Laboratory, Cold Spring Harbor, N.Y. pp. 445-470, 1981; Broach, Cell 28:203-204, 1982; Dilon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980.

A further embodiment of the invention comprises a method of rapidly subcloning a nucleic acid sequence. The invention method comprises contacting a site-specific recombinase and a cell-free solution comprising a donor vector comprising a transfer sequence flanked by a site-specific recombination sequence recognized by the recombinase, and an acceptor vector comprising at least one site-specific recombination sequence recognized by the recombinase, under conditions suitable to promote the transfer of the transfer sequence from the donor vector to the acceptor vector. The invention method employs vectors and recombinases as described above. Means of identifying conditions for the transfer of a transfer sequence from a donor vector to an acceptor vector can readily be determined by those of skill in the art. Suitable conditions include those described in Nunes-Düby et al., EMBO J. 13(18):4421-4430, 1994; Senecoff et al., PNAS USA 82:7270-7274, 1985; Shaikh and Sadowski, J. Biol. Chem. 272(9):5695-5702, 1997; and Peterson and Shuman, J. Biol. Chem. 272(7):3891-3896, 1997, all of which are incorporated by reference herein, and are described in detail in the Examples set out below.

For example, the invention method can be used to perform subcloning (transfer of a DNA or RNA sequence from one vector to another) without PCR amplification using topoisomerase, as described in Examples 1A-C below. In this embodiment of the invention, donor vector is constructed as shown in FIG. 1 with recognition sites for vaccinia topoisomerase I flanking the insertion point for the transfer sequence. The two recognition sites are juxtaposed on opposite strands of the DNA and are generally separated by about four spacer nucleotides to provide overhang. The spacer nucleotides have identical sequences on either side of the insertion point for the gene of interest. One or more vectors is prepared as a linear, double-stranded molecule with single strand overhangs that are compatible with the spacer sequences that flank the gene or gene fragment of interest on the donor vector, as shown in FIG. 1B. In addition, the linear acceptor vector DNA has 5′-hydroxyl groups at each end. A marker gene sequence and additional sequences are included in the acceptor vector as known in the art depending upon the particular attribute of the vector desired. Multiple acceptor vectors useful for different cloning tasks can be simultaneously prepared by including in each those attributes suitable to the task for which the vector would be used.

The donor vector(s) are treated with topoisomerase I for five minutes at room temperature. The enzyme generates nicks at each topoisomerase recognition site, creating double strand breaks at the sites that flank the inserted gene or gene fragment of interest and releasing the transfer DNA fragment. Topoisomerase I is covalently attached at each end of the freed DNA fragment, which also has overhangs complementary to the spacer nucleotides. The topoisomerase treated vector is combined with the linearized acceptor vector in a suitable medium. The compatible ends of each vector corresponding to the spacer sequence brings the two DNA fragments together and allows the topoisomerase I to ligate the spacer sequences together in an ATP independent ligation. The recombinant vector formed, shown in FIG. 1C, contains the gene or gene fragment of interest and can be identified following transformation of the vector into competent E. coli by expression of the marker gene. When Cre or Flp is used as the site specific recombinase, the donor and acceptor vectors are prepared as described in Example 1 except that the recognition sites appropriate to the recombinase of choice flank the insertion point for the gene of interest.

In another embodiment of the invention method, a gene or gene fragment donor clones are created by PCR amplification cloning using primers designed for the specific fragment of interest. A donor vector is not needed. The gene or gene fragment of interest is generated repeatedly from the donor clone for insertion into any or all of the acceptor vectors for a wide variety of research or production applications. No subcloning is required in this technique to move the gene of interest from one vector into another. The gene or gene fragment is simply copied from a donor clone, and the copies are inserted into a “copy ready vector” using the following procedure.

In this procedure, the exact sequence of the open reading frame, if any, and of native features of the gene to be transferred should be noted if the gene is to be expressed as a fusion protein from one or more of the acceptor vectors. For example, signal sequences for intracellular organelle targeting, secretion, glycosylation, etc. are identified in the transfer sequence to determine that the gene of interest is in reading frame with any signal sequence or genes encoding a tag, and the like, in the acceptor vector.

Oligonucleotides are designed for PCR amplification of the exact DNA sequence to be transferred to the acceptor vector(s) using one or more methods well known in the art. For example, to transfer a complete open reading frame, the sequence of one oligonucleotide would have the translation initiation codon at its 5′-end and the sequence of the other oligonucleotide would have the translation initiation codon at its 5′-end. The sequence of the other oligonucleotide would have the complement of the translation termination codon at its 3′-end. Acceptor vectors are prepared as described in Example 1, such as an acceptor vector including DNA sequences appropriate for the expression or analysis of the protein encoded by the gene of interest.

The gene sequence of interest is amplified from the donor clone using the PCR primers prepared as above-described, with cycling parameters selected as suitable for the primer and the template. A 7 to 30 minute extension at 72° C. is optionally included to ensure that all amplified products are full length and 3′ adenylated. The amplified DNA fragment is ligated into the acceptor vector(s). In general, 0.5 to 2 μl of the PCR product (10 ng/μl) with an average insert length of 400 to 100 bp gives a proper insert:vector ratio. Therefore the PCR product is ligated into the acceptor vector by placing 0.5 to 2 μl of PCR product reaction in sterile water to provide a final volume of 4 μl. To this mixture is added 1 μl of the acceptor vector to obtain a final volume of 5 μl., mixing gently and incubating for 5 minutes at room temperature (^{˜}25° C.), then centrifuging briefly and placing the tube on ice. Competent cells, such as E. coli, are then immediately transformed with the acceptor vector(s).

In yet another embodiment of the invention method, gene or gene fragment clones are created by PCR amplification using primers designed specifically, or non-specifically, for the fragment, but which also contain sequences that, when the amplified gene fragment is inserted into an invention donor vector, will allow use of a universal donor vector primer set to create copies of the gene or gene fragment for insertion into one or more specialty application acceptor vectors using the following procedure. If a collection of genes are to be transferred, each gene of interest should be available on a donor plasmid vector and flanked by short sequences that are common to all donor plasmids in the collection. Oligonucleotides for PCR amplification of the gene(s) are synthesized based on the short sequence that flanks each of the transfer sequences in the donor vectors.

An invention acceptor vector containing a recombinase recognition site appropriate for the expression or analysis of the gene of interest is selected. For example, the acceptor vector containing a topoisomerase I recognition site, a strong mammalian promoter, and the coding sequence for an epitope tag would be appropriate for production and analysis of the protein of interest, such as the TOPO Cloning™ vector (Invitrogen, Carlsbad, Calif.). The transfer sequence(s) of interest are amplified from the donor vector using the PCR primers with cycling parameters suitable for the particular primers and template. It may be necessary to include a 7 to 30 minute extension of 72° C. to ensure that all amplified products are full length and 3′ adenylated. The amplified DNA fragments are individually transferred into acceptor vectors) using the insert:vector ratio and conditions described above.

In a presently preferred embodiment of the invention method, the PCR primers add the following sequences at the 5′ end to add topoisomerase I recognition sites to the ends of the amplified PCR product:

| | | |
|---|---|---|
| Forward Primer | 5′-AAGGG | (SEQ ID NO:14) |
| Reverse Primer | 5′-CCCTT | (SEQ ID NO:1) |

The acceptor vector is prepared as a linear molecule with single 3′-T overhangs and 5′-hydroxyl groups. After amplification by PCR, the PCR product is treated with topoisomerase I so that the enzyme becomes covalently bound to each end of the amplified PCR product. Then the covalently bound PCR product is introduced into the acceptor vector(s) as described above.

In another embodiment, the invention provides kits comprising one or more containers or vials containing components for carrying out the methods of the present invention. For instance, such a kit can comprise a suitable reaction solution, recombinase and cells. Also included in the kit are one or more vectors, e.g., vectors for expression in mammalian, bacterial, yeast and insect cells. In a preferred embodiment, the kit will comprise a reaction solution of 50 mM Tris HCl pH 7.5, one or more of the invention vectors that have vaccinia DNA topoisomerase covalently bound thereto, and instructions for their use as described herein.

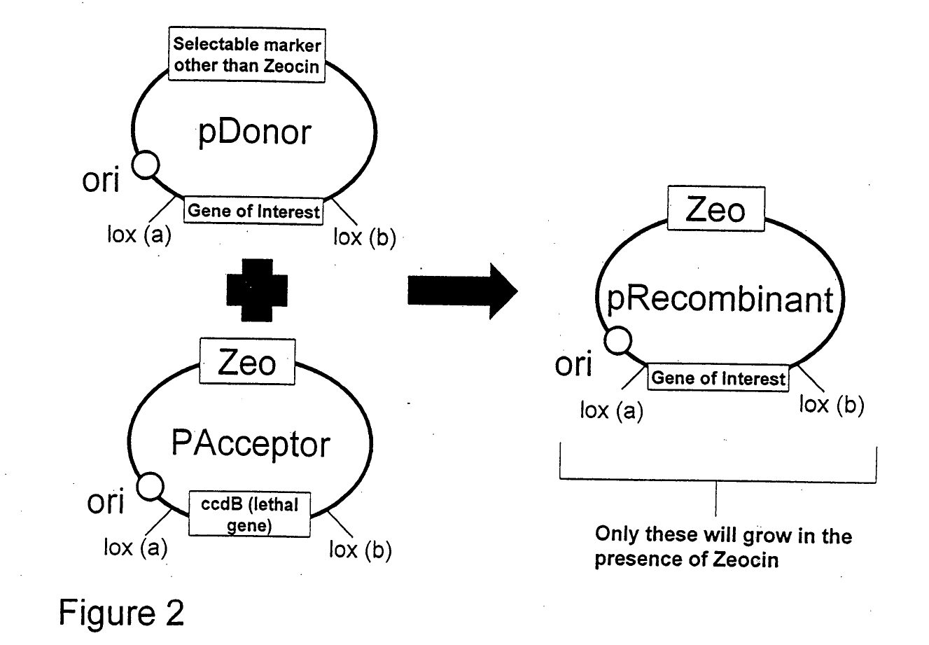

In one embodiment the invention kit comprises at least one donor vector comprising at least one site specific recombination sequence, a transfer sequence, and a first selectable marker, and at least one acceptor vector comprising at least one site specific recombination sequence, a lethal gene and a second selectable marker. For example, as illustrated in FIG. 2, the donor vector in the kit can contain a selectable marker gene other than Zeocin, an origin of replication sequence (“ori”), and a transfer sequence (“gene of interest”) flanked by lox P recognition sites. The acceptor vector (“pAcceptor”) then contains a gene encoding resistance to the antibiotic Zeocin™ (“Zeo”), an origin of replication sequence (“ori”), and a gene encoding ccdB, a lethal compound, flanked by loxP sites. When the donor and acceptor vectors are combined in a reaction mixture in the presence of the recombinase Cre, a new recombinant vector (“pRecombinant”) is created, which recombinant vector contains the transfer sequence and a gene encoding Zeo. Cells transformed with the reaction mixture will grow in the presence of the antibiotic Zeocin™ only if the recombination event has successfully occurred.

Vaccinia DNA topoisomerase can be prepared for expression in E. coli and purified as described in S. Shuman et al., J. Biol. Chem. 263:16401-16407, 1988.

The invention will now be described in greater detail by reference to the following non-limiting Examples.

### EXAMPLE 1

Subcloning without PCR Amplification Using Topoisomerase

Donor vectors can be constructed such that recognition sites for topoisomerase, or other ATP independent enzymes, flank the transfer sequence. In the presence of acceptor vector and topoisomerase, or other ATP independent enzyme, the transfer sequence is occasionally subcloned from the donor vector to the acceptor vector in an ATP independent event.

A linear activated vector containing vaccinia topoisomerase recognition sites (e.g., pCR2.1-TOPO (Invitrogen)) is prepared to receive the transfer sequence. The transfer sequence is amplified from a DNA template of choice. The DNA template may be genomic DNA, plasmid DNA, cosmid DNA or any other shuttle construct. Isolation methods are available in the public domain (Ausubel et al., Section 2.14). Specific oligos (primers) for PCR corresponding to the exact sequence of the transfer DNA are synthesized according to published protocols (Ausubel et al., Section 2.11). Both primers contain 7-9 additional bases on the 5′ ends including the complement to the vaccinia topoisomerase I recognition site (5′-AAGGG 3′) and an additional 2-4 bases which will serve as the 5′ overhangs during subcloning with topoisomerase (5′-CGAAGGG . . . 3′, SEQ ID NO:15). PCR amplification is performed utilizing methods optimized for the template and primers (Ausubel et al., Section 15.1) with a DNA polymerase containing terminal transferase activity, such as Taq (Boehringer Mannheim, Indianapolis, Ind.).

Approximately 20 ng of PCR product is combined with 1 μl of the prepared activated vector in a total volume of 5 μl. The reaction is incubated at 25° C. for 5 min, placed on ice and 1 μl is transformed into competent E. coli using either chemical transformation or electroporation techniques (Ausubel et al., Section 1.8). Transformed cells are plated on appropriate antibiotic selection plates and grown at 37° C. for 12-18 hours. Resulting colonies are screened by miniprep and restriction digest (Ausubel et al., Sections 1.6 and 3,1) to identify clones containing transfer sequence.

Positive clones will contain the transfer sequence flanked on each side by 2 tandem topoisomerase recognition sites on complementary strands separated by 2-4 bases (for example, a direct repeat of 5′-CCCTTGCAAGGG (SEQ ID NO:16) with an intervening transfer sequence). A positive clone is propagated in E. coli and the plasmid DNA is purified as described above. The plasmid DNA is resuspended in TE Buffer, pH 8 (10 mM Tris, 1 mM EDTA) at a concentration of 10 ng/μl. This vector will serve as the donor for subcloning in an ATP independent reaction using topoisomerase.

B. Preparation of the Acceptor Vector

Preparation of linear, dephosphorylated vector: Supercoiled plasmid DNA to be used for construction of the acceptor vector is propagated and purified as described above. The plasmid chosen to be the acceptor vector must have a different E. coli antibiotic selection marker from the donor vector, for example Zeocin™. Plasmid DNA is digested with a restriction enzyme that is unique within the vector and will leave the desired 2-4 base 5′ overhangs (e.g., digestion with BstB I will leave 2 base 5′ overhangs). It is possible to digest with two different enzymes for directional cloning, however the forward and reverse PCR primers used to create the donor vector must be designed to generate the necessary complementary overhangs.

Plasmid DNA (30 μg) is digested with 120 units of BstB I (New England BioLabs, Beverly, Mass.) for 2 hours under conditions specified by the supplier, extracted with an equal volume of phenol/chloroform/isoamyl alcohol (25:24:1), ethanol precipitated, and washed with 500 μl of 80% ethanol (Ausubel et al., Section 2.1). The DNA ends are dephosphorylated by treating with calf intestinal alkaline phosphatase (CIP; New England BioLabs, Beverly, Mass.) according to protocol specified by the supplier, extracted with phenol/chloroform/isoamyl alcohol (25 24:1), ethanol precipitated, and washed with 80% ethanol (Ausubel et al. Section 2.1). The DNA is resuspended in 1000 μl of TE buffer, pH 8.

C. Subcloning with Topoisomerase

Cell-free subcloning and selection: 10 ng of prepared donor vector, 30 ng of prepared acceptor vector and 1 μg of purified topoisomerase are combined in a total volume of 5 μl, and incubated for 5 min. at 25° C. to allow transfer of the desired sequence from the donor vector to the acceptor vector in an ATP independent reaction, The reaction mixture is placed on ice and 1 μl is transformed into competent E. coli using either chemical transformation or electroporation techniques (Ausubel et al., Section 1.8). Clones containing acceptor vector plus transfer sequence are selected by plating on antibiotic media requiring a resistance marker specific to the acceptor vector (e.g., Zeocin™). Plates are incubated at 37° C. for 12-18 hours. Resulting colonies are screened by miniprep and restriction digest (Ausubel et al., Sections 1.6 and 3.1) to identify clones containing the desired transfer sequence subcloned into the acceptor vector.

### EXAMPLE 2

Protocol 2: Subcloning without PCR Amplification Using Site-Specific Recombinases

A. Preparation of Donor Vector

Construct Design: A donor vector is constructed so that a transfer sequence and a unique bacterial selection marker (e.g. Zeocin™, Invitrogen Corp., Carlsbad, Calif.) are flanked by tandemly repeated recombinase recognition sites (for example loxP or FRT). The donor vector construct containing recombinase recognition sites is built using standard molecular biology techniques of PCR and subcloning (Ausubel et al., Sections 3.16 and 3.17). The desired transfer sequence may be subcloned into the donor vector using either standard PCR/restriction digest and ligation techniques (Ausubel et al., Sections 3.16 and 3.17) or by topoisomerase mediated cloning of PCR products as described in Examples 3 and 5 hereafter.

Donor Vector Preparation: The donor plasmid DNA is propagated in E. coli (see Example 1: Section A) and purified from 100 ml of a saturated culture according to protocols specified for the SNAP™ Midiprep Kit (Invitrogen, Carlsbad, Calif.). The plasmid DNA is resuspended in TE Buffer, pH 8 (10 mM Tris, 1 mM EDTA) at a concentration of 0.5 μg/μl.

B. Preparation of the Acceptor Vector

Construct Design: The acceptor vector contains a single recombination recognition site in the desired cloning region that is identical to the two sites on the donor vector. It also contains a bacterial selection marker that differs from that of the donor vector (e.g., Ampicillin) to allow for selection of acceptor vector clones. The acceptor vector is built using standard molecular biology techniques of PCR and subcloning (Ausubel et al., Sections 3.16 and 3.17).

Acceptor Vector Preparation: The acceptor plasmid DNA is propagated in E. coli (Example 1: Section A above) and purified from 100 ml of a saturated culture according to protocols specified for the SNAP™ Midiprep Kit (Invitrogen, Carlsbad, Calif.). The plasmid DNA is resuspended in TE Buffer pH 8 at a concentration of 0.51 μg/μl.

C. Subcloning with a Site-Specific Recombinase

Recombinase (Cre) Reaction: A combination of 0.25 μg of donor vector, 0.75 μg of acceptor vector, 6 μl of 10× Cre Buffer (50 mM Tris-HCl, pH 7.5, 33 mM NaCl, 10 mM MgCl₂, 100 μg/ml BSA) and 2 units of Cre Recombinase (Novagen, Madison, Wis.) is prepared in a 60 μl total volume and incubated at 37° C. for 15 min. Competent E. coli are transformed with 2 μl of the combination using either chemical transformation or electroporation techniques (Ausubel et al., Section 1.8). Based on incompatibility of different vectors containing the same origin of replication within a single cell (Molecular Cloning, A Laboratory Manual, Second Edition, Ed. Sambrook et al., Cold Spring Harbor Laboratory Press, New York, 1989, p. 1.4), clones containing acceptor vector plus transfer sequence are selected by plating on antibiotic media requiring resistance markers specific to both the acceptor vector and the donor vector region that is subcloned (e.g., Ampicillin and Zeocin™). Plates are incubated at 37° C. for 12-18 hours. The resulting colonies are screened by miniprep and restriction digest (Ausubel et al., Sections 1.6 and 3.1) to identify clones containing the desired transfer sequences and subcloned into the acceptor vector.

### EXAMPLE 3

Cloning PCR Amplified DNA with Gene Specific Primers and Cloning Vector.

Gene or gene fragment amplimers are created by PCR amplification using primers sequence-specific to the gene or gene of interest. Any region of DNA containing the gene of interest (designated the donor) and primers specific to the gene of interest can be used to generate the amplimer repeatedly for insertion into any or all of the acceptor vectors for a wide variety of research or production applications. No subcloning is required in this technique to transfer the gene or gene fragment of interest into the acceptor vector. The amplimer is simply copied off from the donor and the copies inserted into the acceptor vector using the procedure described below.

The DNA template should be available in sufficient quantities (at least 20 ng for plasmids) and the complete sequence of the target open reading frame should be known. DNA template may be genomic DNA, plasmid DNA, cosmid DNA or any other shuttle construct. Isolation methods are those known in the art, for example, as disclosed in Ausubel et al., Section 2.14.

Specific oligonucleotides (primers) for PCR corresponding to the exact DNA sequence to be transferred to the acceptor vector are prepared. For example, to transfer a complete open reading frame, the sequence of the 5′ primer would contain the translation initiation codon and flanking sequences of the target sequence. The sequence of the 3′ primer would contain the complement of the translation termination sequence of the target. Protocols describing the synthesis of oligonucleotides are available in the public domain (Ausubel et al., Section 2.11).

An acceptor vector appropriate for the expression or analysis of the gene or gene fragment of interest is TOPO Cloning™ vector, having the topoisomerase already associated with the linear plasmid, for example, pCR2.1TOPO™ (Invitrogen,

The transfer sequence of interest is obtained from the donor clone in a 50 μl reaction volume using the PCR primers specific to the transfer sequence. Cycling parameters are selected to be appropriate for the primers and template used (Ausubel et al., Section 15.1). It may be necessary to include a 7 to 30 minute extension at 72° C. after PCR is complete to ensure that all amplimers are full length and 3 adenylated (Ausubel et al., Section 15.7).

The amplimer is cloned into the acceptor vector as follows: For one reaction, 0.5 to 2 μl fresh PCR product is combined with 1 μl of the acceptor vector and sterile water is added to a 5 μl total volume. The mixture is gently stirred and incubated for 5 minutes at room temperature (˜25° C.) and then competent E. coli cells are immediately transformed with the mixture by any known method.

In general, 0.5 to 2 μl of a typical PCR reaction (10 ng/μl) with an average amplimer length of 400 to 1000 bp will give the proper insert:vector ratio.

### EXAMPLE 4

Cloning PCR Amplified DNA with Generic Primers and a Cloning Vector.

Gene or gene fragment amplimers are created by PCR amplification using primers of sequence specific to the donor vector and unrelated to the transfer sequence (generic). Any plasmid containing the transfer sequence (designated the donor plasmid) and primers specific to the donor plasmid can be used to generate the amplimer repeatedly for insertion into any or all of the acceptor vectors for a wide variety of research or production applications. No subcloning is required in this technique to transfer the gene or gene fragment of interest into the acceptor vector. The amplimer is simply copied off from the donor plasmid and the copies inserted into the acceptor vector using the procedure described below.

The donor plasmid should be available in sufficient quantities (at least 20 ng) and the complete sequence of the target open reading frame should be known. Isolation methods are well known in the art (Ausubel et al., Section 2.14).

Specific oligonucleotides (primers) are prepared corresponding to the plasmid DNA sequences flanking the amplicon to be transferred to the acceptor vector. Primers need to be made corresponding to regions of the plasmid immediately upstream and downstream of the amplicon. Protocols describing the synthesis of oligonucleotides are well known in the art (Ausubel et al., Section 2.11).

An acceptor vector appropriate for the expression or analysis of the gene or gene fragment of interest and having the topoisomerase already associated with the linear plasmid is prepared. Such vectors are commercially available as TOPO Cloning™ vector, having the topoisomerase already associated with the linear plasmid, for example, pCR2.1 TOPO™ (Invitrogen, Carlsbad, Calif.).

The transfer sequence is cloned from the donor plasmid in a 50 μl reaction volume using the PCR primers specific to the donor plasmid, and utilizing cycling parameters that are appropriate for the primers and template as described in Example 3 above. The amplimer is cloned into the acceptor vector as described in Examples 1-3 above.

### EXAMPLE 5

Transferring PCR Amplified DNA Treated with Topoisomerase.

A desired transfer sequence is amplified from a donor clone by PCR using primers specific for the transfer sequence. The inclusion of topoisomerase recognition sites at the 5′ ends of the PCR primers enables transfer of the amplified sequence to an appropriate acceptor vector when treated with topoisomerase.

A. PCR Amplified Transfer DNA

Preparation of amplified transfer DNA treated with topoisomerase: A donor clone may be genomic DNA, cDNA, plasmid DNA, cosmid DNA or any other shuttle construct. DNA from the donor clone is prepared for use as a template in, PCR amplification utilizing an appropriate preparation technique (Ausubel et. al., Sections 2.11 and 5.5). The sequence of the transfer DNA is known. DNA PCR primers containing the complement of the vaccinia topoisomerase I recognition site (SEQ ID NO:14 followed by transfer DNA specific sequence are synthesized according to known protocols (Ausubel et. al, Section 2.11). The DNA fragment generated using these primers will contain topoisomerase recognition sites at the 3′ ends. An additional 2-4 bases may be added at the 5′ ends of each primer to create 5′ overhangs in the amplified DNA after treatment with topoisomerase. For example, including SEQ ID NO:14 in the primer will result in 5′ overhangs complementary to those generated by digestion with EcoR 1).

The transfer sequence is amplified by PCR following established methods (Ausubel et. al, Section 15.1). 200 ng of amplification product, 200 ng of purified topoisomerase I and TE buffer, pH 8 (10 mM Tris, 1 mM EDTA) are combined in a total volume of 20 μl. The reaction is incubated at 25° C. for 5 min and placed on ice. The topoisomerase will be covalently bound to the 3′ ends of the PCR product, leaving the desired 5′ overhangs.

B. Preparation of the Acceptor Vector

Preparation of linear, dephosphorylated vector: Supercoiled plasmid DNA to be used for construction of the acceptor vector is propagated and purified as described previously (Example 1, Section A). The plasmid chosen to be the acceptor vector should have a different E. coli antibiotic selection marker from the donor vector. Plasmid DNA is digested with a restriction enzyme that is unique within the vector and will leave the desired 2-4 base 5′ overhangs (e,g. digestion with EcoR I will leave 4 base 5′ overhangs: 5′-AATT . . . -3′). It is possible to digest the acceptor vector with two different enzymes for directional cloning, however the forward and reverse PCR primers used to create the amplified transfer DNA must be designed to generate the necessary complementary overhangs.

The supercoiled DNA of the acceptor vector is digested with 120 units of EcoR 1 (New England BioLabs, Beverly, Mass.) for 3 hours under conditions specified by the supplier, extracted with an equal volume of phenol/chloroform/isoamyl alcohol (25:24:1), ethanol precipitated, and washed with 500 μl of 80% ethanol (Ausubel et. al, Section 2.1). Ends of the DNA are dephosphorylated by treating with calf intestinal alkaline phosphatase (CIP; New England BioLabs, Beverly, Mass.) according to protocol specified by the supplier, then the DNA is extracted with phenol/chloroform/isoamyl alcohol (25:24:1), ethanol precipitated, washed with 80% ethanol, and resuspended in 1000 μl of TE buffer, pH 8.

C. DNA Sequence Transfer:

Cloning the PCR amplified product into the acceptor vector: A combination of 4 μl (40 ng) of the topoisomerase treated PCR product (400 bp-2000 bp) and 1 μl (30 ng) of the prepared acceptor vector is prepared and incubated at 25° C. for 5 min., the reaction is placed on ice, and then 1 μl of the combination is transformed into competent E. coli using either chemical transformation or electroporation techniques (Ausubel et. al, Section 1.8). Cells containing the acceptor vectors plus transfer sequence are selected by plating on antibiotic media requiring a resistance marker specific to the acceptor vector. Plates are incubated at 37° C. for 12-18 hrs. and resulting colonies are screened by miniprep and restriction digest (Ausubel et. al, Sections 1.6 and 3.1) to identify acceptor vector clones containing the desired transfer sequence.

While the foregoing has been with reference to particular embodiments of the invention, it will be appreciated by those skilled in the art that changes in these embodiments may be made without departing from the principles and spirit of the invention, the scope of which is defined by the appended claims.

1-26. (canceled)

27. An in vitro method of cloning a nucleic acid molecule comprising:
(a) obtaining a first nucleic acid molecule to be cloned;
(b) mixing said first nucleic acid molecule to be cloned in vitro with a second nucleic acid molecule comprising at least a first topoisomerase recognition site flanked by at least a first recombination site, and at least a second topoisomerase recognition site flanked by at least a second recombination site, wherein said first and second recombination sites do not recombine with each other, and at least one topoisomerase; and
(c) incubating said mixture under conditions such that said first nucleic acid molecule to be cloned is inserted into said second nucleic acid molecule between said first and second topoisomerase recognition sites, thereby producing a first product molecule comprising said first nucleic acid molecule to be cloned between said first and second recombination sites.

28. The method of claim 27, wherein the second nucleic acid molecule is a vector.

29. The method of claim 27, wherein said first nucleic acid molecule to be cloned is a linear nucleic acid molecule.

30. The method of claim 29, wherein said linear nucleic acid molecule is a blunt-end nucleic acid molecule.

31. The method of claim 27, wherein said first nucleic acid molecule to be cloned is a PCR product.

32. The method of claim 27, wherein said first nucleic acid molecule to be cloned comprises at least one open reading frame.

33. The method of claim 27, further comprising contacting said first product molecule with at least one third nucleic acid molecule comprising at least a third and fourth recombination sites that do not recombine with each other, under conditions favoring recombination between said first and third and between said second and fourth recombination sites, thereby producing at least one second product molecule.

34. The method of claim 33, wherein the third nucleic acid molecule is a vector.

35. The method of claim 27, further comprising inserting said first product molecule into a host cell.

36. The method of claim 28, further comprising inserting said first product molecule into a host cell.

37. The method of claim 33, further comprising inserting said second product molecule into a host cell.

38. The method of claim 34, further comprising inserting said second product molecule into a host cell.

39. The method of claim 28, wherein said vector is an expression vector.

40. The method of claim 34, wherein said vector is an expression vector.

41. The method of claim 27, wherein said second nucleic acid molecule comprises at least one additional nucleic acid sequence selected from the group consisting of a selectable marker, a cloning site, a restriction site, a promoter, an operator, an operon, an origin of replication, and a gene or partial gene.

42. The method of claim 33, wherein said third nucleic acid molecule comprises at least one additional nucleic acid sequence selected from the group consisting of a selectable marker, a cloning site, a restriction site, a promoter, an operator, an operon, an origin of replication, and a gene or partial gene.

43. The method of claim 27, wherein said first and second recombination sites are selected from the group consisting of:
(a) attB sites,
(b) attP sites,
(c) attL sites,
(d) attR sites,
(e) lox sites,
(f) psi sites,
(g) dif sites,
(h) cer sites,
(i) frt sites,
and mutants, variants, and derivatives of the recombination sites of (a), (b), (c), (d), (e), (f), (g), (h) or (i) which retain the ability to undergo recombination.

44. The method of claim 33, wherein said third and fourth recombination sites are selected from the group consisting of:
(a) attB sites,
(b) attP sites,
(c) attL sites,
(d) attR sites,
(e) lox sites,
(f) psi sites,
(g) dif sites,
(h) cer sites,
(i) frt sites,
and mutants, variants, and derivatives of the recombination sites of (a), (b), (c), (d), (e), (f), (g), (h) or (i) which retain the ability to undergo recombination.

45. The method of claim 43, wherein said lox sites are selected from the group consisting of loxP sites and loxP511 sites.

46. The method of claim 44, wherein said lox sites are selected from the group consisting of loxP sites and loxP511 sites.

47. The method of claim 27, wherein said topoisomerase is a type I topoisomerase.

48. The method of claim 33, wherein said product nucleic acid molecule and said third nucleic acid molecule are combined in the presence of at least one recombination protein.

49. The method of claim 48, wherein said recombination protein is selected from the group consisting of:
(a) Cre;
(b) Int;
(c) IHF;
(d) Xis;
(e) Fis;
(f) Hin;
(g) Gin;
(h) Cin;
(i) Tn3 resolvase;
(j) TndX;
(k) XerC; and
(l) XerD.

50. The method of claim 48, wherein said recombination protein is Cre.

51. The method of claim 48, wherein said recombination protein is selected from the group consisting of Int, Xis, IHF and Fis.
